(19) United States
(12) Patent Application Publication
(10) Pub. No.: US 2020/0211690 A1
(43) Pub. Date: Jul. 2, 2020

(54) INFORMATION PROTECTION LABEL, AND PRODUCT WITH THE SAME PASTED THEREON

(71) Applicant: LINTEC CORPORATION, Tokyo (JP)

(72) Inventors: Kentaro MIYAZAKI, Tokyo (JP); Katsuhiro ARAKI, Tokyo (JP); Fuminori IKEDA, Tokyo (JP)

(21) Appl. No.: 16/716,820

(22) Filed: Dec. 17, 2019

(30) Foreign Application Priority Data
Dec. 27, 2018 (JP) 2018-245989

(51) Int. Cl. G16H 20/13

(52) U.S. Cl. CPC G16H 20/13

(57) ABSTRACT

An information protection label in which information becomes visually unrecognizable by a simple process, and a product with the information protection label pasted thereon are disclosed. The information protection label (1) comprises a base material layer (4) comprising an information display part (2) for displaying information and a base material (3), and an adhesive layer (5). The base material layer (4) comprises a material which changes in quality so that the information becomes visually unrecognizable by an autoclave process. The product with the information protection label (1) pasted thereon is preferably a biological sample container, a blood transfusion bag, an infusion bottle, a medical container such as a box containing a medicine, or a medical device, which are used for academic research, examination, treatment or the like.

### TECHNICAL FIELD

The present inventions relate to an information protection label, and a product with the information protection label pasted thereon.

### BACKGROUND ART

In many cases, a label pasted on a surface of a product or a product package on which the product related information is written, describes contents which an individual does not want to be seen by other people, such as personal information such as a name, an address, and a telephone number of the individual, as well as information such as an order detail, a delivery notice and an invoice.

In particular, a label specifying personal information such as a blood type and a name of an individual and medical information such as a disease name and a medicine name, are generally pasted on a product in the biomedical field such as a biological sample container, a medical container and a medical device, in order to prevent misunderstanding of samples and misadministration (see Patent Literature 1). When other people see the information specified on the label, there is a problem that it is easy to guess what kind of diseases the individual suffers from, which is not preferable from a viewpoint of personal information protection.

As a method of protecting information on a label, measures are taken such as removing the label from the product or package, followed by cutting the label with scissors or a shredder, and painting the part indicating the information on the label, followed by discarding the label. However, such measures are laborious. There is a risk that the information can be speculated from the discarded label, if the measures are insufficient. Further, there is a risk that infectious wastes may be touched during the removing process.

As a solution to this problem, for example, Patent Literature 2 discloses a personal information protection sheet having a base material and a releasing layer formed on at least a part of the surface of the base material from which an information display part formed thereon can be removed.

Patent Literature 3 discloses a label specifying medical information of a medicine and personal information of a patient to administer the medicine, which is to be pasted on a container filled with the medicine or a container for infusion prepared by adding the medicine when used; wherein the label comprises a main display part and an auxiliary display part whose back sides are adhesive surfaces, and a personal information display part formed between the main display part and the auxiliary display part so as to be separable from the main display part and the auxiliary display part, a part or all of whose back sides is a non-adhesive surface; wherein a cutoff line is formed on the boundary between the personal information display part and the main display part and the auxiliary display part; wherein at least one of the main display part and the auxiliary display part displays the information of the medicine; and wherein the personal information is displayed only on the personal information display part.

### CITATION LIST

### Patent Literature

Patent Literature 1: JP H08-141050

Patent Literature 2: JP 2006-281764

Patent Literature 3: JP 2008-033115

### SUMMARY OF THE INVENTION

### Technical Problem

However, regarding the personal information protection sheet described in Patent Literature 2, the personal information formed on the releasing layer can be removed by scratching the releasing layer with a coin or a nail. But, the removed releasing layer is a garbage, and its cleaning process is necessary. It is not also secure to perform the removing process in accordance with the direction. Therefore, the label described in Patent Literature 2 is still insufficient for protecting leakage of personal information.

In addition, regarding the label described in Patent Literature 3, it is still laborious to separate the personal information display part along a cutoff line. It is not also secure to perform the separation process in accordance with the direction. Therefore, the label described in Patent Literature 3 is still insufficient for protecting leakage of personal information.

Accordingly, the present inventions have been invented so as to solve the above problems. The objects of the present inventions are to provide an information protection label in which information becomes visually unrecognizable by a simple process, and a product which the information protection label is pasted on.

### Solution to the Problem

The present inventors have done intensive studies to solve the above problems. As a result, the present inventors paid attention to a medical container to be discarded through an autoclave sterilization process after its usage, and the present inventors have found that information effectively becomes visually unrecognizable by the base material layer of the information protection label having a material which changes in quality so that the information becomes visually unrecognizable by an autoclave sterilization process (hereinafter, referred simply as “autoclave process”). Thus, the present inventions have been accomplished.

According to the present inventions, the following information protection label, and the product with the information protection label pasted thereon are provided.

[1] The first embodiment of the present inventions is:

an information protection label, comprising:

a base material layer comprising an information display part for displaying information and a base material, and

an adhesive layer,

wherein the base material layer comprises a material which changes in quality so that the information becomes visually unrecognizable by an autoclave process.

[2] The second embodiment of the present inventions is:

the information protection label according to [1],

wherein the base material is a shrinkable film; and

the adhesive layer is composed of an adhesive composition whose adhesive strength is reduced by an autoclave process.

[3] The third embodiment of the present inventions is:

the information protection label according to [1] or [2], wherein the base material develops a dark color or black color by an autoclave process.

[4] The fourth embodiment of the present inventions is:

the information protection label according to any one of [1] to [3], wherein the base material exhibits solubility by an autoclave process.

[5] The fifth embodiment of the present inventions is:

the information protection label according to any one of [1] to [4], wherein the base material is foamed by an autoclave process.

[6] The sixth embodiment of the present inventions is:

the information protection label according to any one of [1] to [5], wherein the information display part is printed with an ink exhibiting a water disintegratability.

[7] The seventh embodiment of the present inventions is:

the information protection label according to any one of [1] to [6], wherein the base material layer comprises an ink receiving layer exhibiting a water disintegratability by an autoclave process.

[8] The eighth embodiment of the present inventions is:

the information protection label according to any one of [1] to [7],

wherein a transparent or translucent laminate base material is provided on the surface of the information display part, and

the laminate base material has a property of developing a dark color or black color by an autoclave process.

[9] The ninth embodiment of the present inventions is:

the information protection label according to any one of [1] to [8], which is used as a medical label.

[10] The tenth embodiment of the present inventions is:

a product which the information protection label according to any one of [1] to [9] is pasted on.

[11] The eleventh embodiment of the present inventions is:

the product according to [10], wherein the product is any one of a biological sample container, a medical container and a medical device.

### Advantageous Effects of the Invention

The present inventions can provide an information protection label in which information becomes visually unrecognizable by a simple process, and a product which the information protection label is pasted on.

### BRIEF DESCRIPTION OF THE DRAWINGS

FIG. 1 shows a schematic cross sectional view of an embodiment of the information protection label of the present inventions.

FIG. 2 shows a schematic cross sectional view of another embodiment of the information protection label of the present inventions.

FIG. 3 shows a schematic cross sectional view of another embodiment of the information protection label of the present inventions.

### DESCRIPTION OF THE INVENTION

Embodiments for performing the present inventions are demonstrated in detail hereinafter.

### First Embodiment

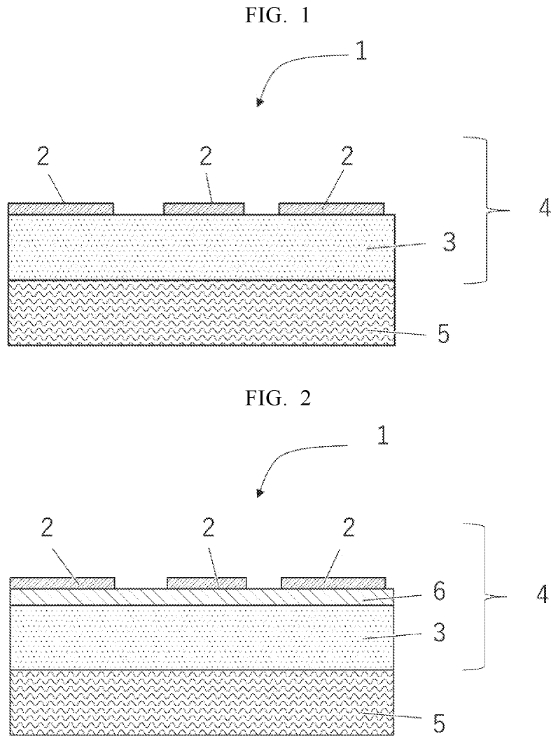

As exhibited in FIG. 1, the information protection label (1) of the present embodiment comprises a base material layer (4) comprising an information display part (2) for displaying information and a base material (3), and an adhesive layer (5).

### (Base Material Layer)

The base material layer (4) comprises a material which changes in quality so that information becomes visually unrecognizable by an autoclave process. The material which changes in quality so that information becomes visually unrecognizable by an autoclave process is not particularly limited, as long as the material is chemically or physically deformable and lets the information become visually unrecognizable. Here, the autoclave process is usually performed by a steam heating and pressurizing process at 121° C. for 20 min, but its condition such as a temperature and a process time can be appropriately selected according to a desired product and a process purpose. A device for the autoclave process is not particularly limited, as long as a steam, a pressure and a temperature can be set and adjusted. A general device such as a commercially available autoclave device, pressure cooker and pressure vessel may be used. The base material layer (4) should just comprise a material which changes in quality so that information becomes visually unrecognizable by the above mentioned autoclave process.

The information protection label (1) of the present invention is the information protection label (1) in which the base material layer (4) comprises a material which changes in quality so that information becomes visually unrecognizable by an autoclave process. By comprising a material which changes in quality so that information becomes visually unrecognizable by an autoclave process, information protection can be acquired simultaneously with the autoclave process, and information becomes visually unrecognizable by a simple process without necessity of separating the information display part (2).

In the information protection label (1) of the present invention, the base material (3) is preferably a material possessing a property of changing in quality so that the information becomes visually unrecognizable by an autoclave process. Specifically, in the information protection label (1) of the present invention, the base material (3) is preferably a shrinkable film, and the adhesive layer (5) is preferably composed of an adhesive composition whose adhesive strength is reduced by an autoclave process. The adhesive strength of the adhesive layer (5) is reduced due to the heating during the autoclave process, and the thermal shrinkage of the shrinkable film is promoted to let the information become visually unrecognizable.

The shrinkable film is not particularly limited, but examples thereof include, for example, a polyester film, a polyolefin film, a polystyrene film, a polyvinyl chloride film, and the like. Among these films, a material containing at least one of a polyester film, a polystyrene film and a polyolefin film is preferable, from the viewpoint of easily exhibiting the desired heat shrinkage. A heat shrinkable polyester film is preferably used from a viewpoint of reduction of cost and environmental load. The heat shrinkable polyester film is particularly preferably a material including polyethylene terephthalate, from a viewpoint of its suitablity for a print label or the like. A commercially available product may be used as the heat shrinkable polyester film, and examples thereof include the longitudinal uniaxial stretched heat shrinkable polyester film “Daiwa Bell Fine (registered trademark)” manufactured by Daiwa Can Company, and the like. The shrinkable film may be a single layer or a multilayer. Moreover, the layer constituting the shrinkable film may be composed of a single resin, or may be composed of plurality of resins. The thickness of the shrinkable film is preferably 10 μm or more, more preferably 20 μm or more, and particularly preferably 30 μm or more. Moreover, the thickness of the shrinkable film is preferably 80 μm or less, and particularly preferably 50 μm or less. In case that the thickness of the shrinkable film is within the above range, both a handling property of the information protection label (1) and a property of changing in quality so that the information becomes visually unrecognizable by an autoclave process, can be easily achieved.

A thermal shrinkage rate of the shrinkable film is preferably 10% or more, more preferably 20% or more in the MD direction and/or the CD direction after autoclave sterilization process at 121° C. for 20 min. Here, the MD direction refers to the flow direction in extrusion molding, which is one of the shrinkable film production processes, and the CD direction refers to the direction orthogonal to the MD direction. In case that the thermal shrinkage rate of the shrinkable film is within the above range, the information easily becomes visually unrecognizable by an autoclave process. The shrinkable film may be uniaxially stretched or biaxially stretched.

A heat shrinkage rate of the shrinkable film is measured by the following measuring method. For the label in which the base material layer (4) is composed of a shrinkable film, each four pieces of samples having a width of 10 mm×a length of 120 mm in which the MD direction is the longitudinal direction and samples having a width of 10 mm×a length of 120 mm in which the CD direction is the longitudinal direction, are prepared respectively. For each sample, a standard line with a length of 100 mm parallel to the longitudinal direction is drawn and used as an evaluation sample. The evaluation sample is subjected to an autoclave sterilization process at 121° C. for 20 min. The evaluation sample taken out from the autoclave is left to stand at room temperature (25° C.), and then the length of the standard line of 100 mm parallel to the longitudinal direction (LMD) is measured again. The shrinkage rate in each direction is measured using the following formula.

Shrinkage rate (%)=[(100−LMD)/100]×100

(Adhesive layer)

The adhesive composition contained in the adhesive layer (5) is preferably composed of an adhesive composition whose adhesive strength is reduced by an autoclave process. Examples of the adhesive composition whose adhesive strength is reduced by an autoclave process, include a rubber based adhesive, a thermosetting adhesive, a water soluble adhesive, and a heat foamable adhesive.

Adhesive strength of the rubber based adhesive is reduced by melting of the rubber based adhesive by the heating during an autoclave process. The rubber based adhesive is preferably a hot melt type adhesive. Examples of the rubber based adhesive include a natural rubber; a modified natural rubber obtained by graft polymerization of one or more monomers selected from a (meth)acrylic acid alkyl ester, styrene and a (meth)acrylonitrile on a natural rubber; a diene homopolymer such as a polybutadiene, a polyisoprene and a polychloroprene; a diene copolymer such as a polystyrene-polybutadiene and a polystyrene-polyisoprene; a styrene-butadiene rubber; an acrylonitrile-butadiene rubber; a methyl methacrylate-butadiene rubber; an urethane rubber; a polyisobutylene resin; a polybutene resin, and the like. The rubber based adhesive may be used alone or in combination of two or more.

Adhesive strength of the thermosetting adhesive is reduced by heat curing by an autoclave process. An example thereof is a thermosetting adhesive obtained by adding a thermosetting agent to a resin having adhesiveness such as a rubber based adhesive and an acrylic adhesive. Examples of the thermosetting agent for the acrylic adhesive include an isocyanate-based crosslinking agent, an epoxy-based crosslinking agent, an aziridine crosslinking agent, and a chelate-based crosslinking agent. The amount of the thermosetting agent is not specifically limited, but is preferably 0.1 parts by weight or more and 20 parts by weight or less, more preferably 2 parts by weight or more and 7 parts by weight or less, with respect to 100 parts by weight of the acrylic adhesive.

Adhesive strength of the water soluble adhesive is reduced by dissolution of the water soluble adhesive by a steam generated during an autoclave process. The water soluble adhesive is not particularly limited as long as it has adhesiveness in a normal state. Examples of the water soluble adhesive include a polyvinyl alcohol based water soluble adhesive, a polyethylene oxide based water soluble adhesive, a polyvinyl amide based water soluble adhesive, a polyvinyl pyrrolidone based water soluble adhesive, a polyvinyl acetal based water soluble adhesive, an acryl based water soluble adhesive, a cellulose resin based water soluble adhesive, a gum arabic based water soluble adhesive, a gelatin based water soluble adhesive, a starch based water soluble adhesive, and the like. Among these, an acryl based water soluble adhesive is preferable. Examples of the acryl based water soluble adhesive include a water soluble adhesive containing an acryl based polymer such as a polymer consisting of a carboxylic acid such as itaconic acid, maleic acid, acrylic acid, methacrylic acid and a derivative thereof, or a salt or an ester of the carboxylic acid, and a polyacrylamide, and the like.

Adhesive strength of the heat foamable adhesive is reduced by reduction of the contact area caused by expansion of the foaming agent by the heating during an autoclave process.

The foaming agent is not particularly limited. For example, an inorganic or organic foaming agent, and thermally expandable microspheres (microcapsules), or the like may be used.

The inorganic foaming agent includes, for example, ammonium carbonate, ammonium hydrogen carbonate, sodium hydrogen carbonate, ammonium nitrite, sodium borohydride, an azide and the like. The organic foaming agent includes, for example, a chlorofluoroalkane compound such as trichloromonofluoromethane and dichloromonofluoromethane; an azo compound such as azobisisobutyronitrile, azodicarbonamide, and barium azodicarboxylate; a hydrazine compound such as p-toluenesulfonyl hydrazide, diphenylsulfone-3,3′-disulfonyl hydrazide, 4,4′-oxybis(benzenesulfonyl hydrazide), allyl bis(sulfonyl hydrazide); a semicarbazide compound such as p-toluelenesulfonyl semicarbazide, 4,4′-oxybis(benzenesulfonyl semicarbazide); a triazole compound such as 5-morpholyl-1,2,3,4-thiatriazole; an N-nitroso compound such as N, N′-dinitrosopentamethylenetetramine, N,N′-dimethyl-N,N′-dinitrosoterephthalamide; and the like. These foaming agents may be used alone or in combination of two or more.

Among these, thermally expandable microspheres are preferable. The thermally expandable microspheres may be microspheres encapsulating in a shell having elasticity a substance which expands by gasification by heating, that is, a hydrocarbon such as propane, propylene, butene, n-butane, isobutane, isopentane, neopentane, n-pentane, n-hexane, isohexane, heptane, octane, petroleum ether and methane; a low boiling point liquid such as tetraalkylsilane; azodicarbonamide to be gasified by thermal decomposition; or the like. These thermally expandable microspheres may be used alone or in combination of two or more.

As a substance forming the shell, for example, a substance exhibiting a heat melting property or a substance that can be ruptured by thermal expansion of an encapsulated substance, may be used. Examples of such substance include a vinylidene chloride-acrylonitrile copolymer, a polyvinyl alcohol, a polyvinyl butyral, a polymethyl methacrylate, a polyacrylonitrile, a polyvinylidene chloride, polysulfone, and the like. These substances may be used alone or in combination of two or more.

The thermally expandable microspheres can be produced by a conventional method such as a coacervation method, an in situ method, an interfacial polymerization method, and the like. Examples of commercially available thermally expandable microspheres include “Matsumoto Microsphere” manufactured by Matsumoto Yushi Seiyaku Co., Ltd., “Expancel” manufactured by Japan Fillite Co., Ltd., “Daiform” manufactured by Dainichiseika Color & Chemicals Mfg. Co., Ltd., “Advancel” manufactured by Sekisui Chemical Co., Ltd. and the like.

The adhesive composition may be used alone or in combination of two or more. Moreover, the adhesive layer (5) constituted by the adhesive composition whose adhesive force is reduced by an autoclave process, may be used, even if the base material layer 4 is not composed of a shrinkable film.

The adhesive composition which is a material for forming the adhesive layer (5), may contain an adhesive additive depending on a type of the adhesive resin and a usage of the information protection label (1). Examples of the adhesive additive include a crosslinking agent, a tackifier, a filler, an antioxidant, a heat stabilizer, an ultraviolet absorber, a light stabilizer, a colorant, a flame retardant, a thickener, an antistatic agent, a polymerization initiator, a curing aid and the like.

The thickness (when dried) of the adhesive layer (5) is preferably 0.1 μm or more and 50 μm or less, and more preferably 2 μm or more and 30 μm or less. In the case that the thickness is less than 0.1 μm, when the information protection label (1) is pasted on the adherend, followability is poor and the information protection label (1) is easily peeled off. On the other hand, in the case that the thickness exceeds 50 μm, the decrease of adhesiveness by the autoclave process is insufficient.

Examples of a method of providing the adhesive layer (5) on the base material layer (4) include a coating method such as an extrusion lamination method, a knife coater method, a roller coating method, a spray method, and the like. The adhesive layer (5) may be directly formed on the base material layer (4) by one of these methods. Or, once the adhesive layer (5) is formed on a peeling sheet, the base material layer (4) is laminated to the peeling sheet at the side of the adhesive layer (5), and the peeling sheet may be removed.

<Adhesive Strength Before and after Autoclave Process and its Ratio>

An adhesive strength (A1) of the information protection label (1) of the present invention to a polypropylene (PP) plate 24 hours after pasting and before an autoclave sterilization process, is preferably 2N/25 mm or more, more preferably 3N/25 mm or more and 30 N/25 mm or less, and further preferably 5 N/25 mm or more and 30 N/25 mm or less. Further, an adhesive strength (A2) of the information protection label (1) to a polypropylene plate (PP) after it is autoclaved at 121° C. for 20 min is preferably 1 N/25 mm or less, and more preferably 0.5 N/25 mm or less.

The ratio ((A2)/(A1)) of the adhesive strengths (A1) and (A2) of the information protection label (1) before and after the autoclave process is preferably 0.0001 or more and 0.5 or less, more preferably 0.0005 or more and 0.1 or less. When the ratio ((A2)/(A1)) of the adhesive strength of the information protection label (1) before and after the autoclave process is 0.0001 or more and 0.5 or less, the adhesive strength of the adhesive layer (5) is reduced due to the heating during the autoclave process so that the label is removed from the adherend, and the thermal shrinkage of the shrinkable film is promoted to let the information become visually unrecognizable.

A method of measuring the adhesive strengths (A1) and (A2) of the information protection label (1) before and behind the autoclave process is demonstrated hereinafter, respectively.

### <(A1) Measurement Method>

Polypropylene (PP) is provided as an adherend. The adhesive surface of a 25 mm×250 mm sample is pasted on the PP under an environment of 23° C. and 50% RH using a roll having a diameter of 95 mm and a mass of 2 kg, the surface of which is coated with a rubber layer. After that, it is allowed to stand for 24 hours under an environment of 23° C. and 50% RH. A value (N/25 mm) is measured under the same environment and under the conditions of a peeling speed of 300 mm/min and a peeling angle of 180° using a tensile testing instrument (product name “Tensilon”, manufactured by Orientec Co., Ltd.), according to the Japanese Industrial Standard: JIS Z 0237. The value is defined as the adhesive strength (A1).

### <(A2) Measurement Method>

Polypropylene (PP) is provided as an adherend. The adhesive surface of a 25 mm×250 mm sample is pasted on the PP under an environment of 23° C. and 50% RH using a roll having a diameter of 95 mm and a mass of 2 kg, the surface of which is coated with a rubber layer. After 24 hours, an autoclave process is performed at 121° C. for 20 min, and then the sample is returned to an environment of 23° C. and 50% RH. A value (N/25 mm) is measured under the same environment and under the conditions of a peeling speed of 300 mm/min and a peeling angle of 180° using a tensile testing instrument (product name “Tensilon”, manufactured by Orientec Co., Ltd.), according to the Japanese Industrial Standard: JIS Z 0237. The value is defined as the adhesive strength (A2).

### (Information Display Part)

The information display part (2) displays various information such as an individual's name, address and telephone number, a date, a content display, a blood type, a barcode, a QR code (registered trademark), and the like. In the present invention, the information display part (2) includes not only one in a state where information is displayed, but also one in a state where information can be displayed. The information display part (2) displays information by printing, typing, writing, or the like.

### (Peeling Sheet)

In the information protection label (1) of the present invention, a peeling sheet can be provided on the adhesive layer (5) as necessary. Examples of this peeling sheet includes paper such as kraft paper, glassine paper and high-quality paper; paper obtained by coating one side or both sides of these paper with a synthetic resin such as polyvinyl alcohol or clay or the like; paper obtained by laminating one side or both sides of these paper with a polyethylene resin or the like; a sheet obtained by coating one side or both sides of a plastic film such as polyethylene terephthalate, polyethylene naphthalate and polypropylene with a release agent such as a fluorine resin and a silicone resin; and the like. The thickness of the peeling sheet is not particularly limited, but is generally in the range of 20 μm or more and 300 μm or less.

### Second Embodiment

Mainly different points from the contents described in the first embodiment are specifically demonstrated hereinafter. In the information protection label (1) of the present invention, the base material (3) preferably has thermosensitive chromogenicity by an autoclave process, and the base material (3) preferably develops a dark color or black color. The base material (3) is colored dark or black due to an autoclave process, so that the information becomes visually unrecognizable.

Examples of the base material (3) which develops a dark color or black color by an autoclave process, include a thermal film, a thermal coloring paper, and the like. Specific examples include one in which at least one thermosensitive coloring layer is formed on the base material (3) at the side of the information display part (2), and the like. One in which at least one thermosensitive coloring layer is formed on the base material (3) at the opposite side to the information display part (2), is also applicable. The thermosensitive coloring layer is formed, for example, by applying on the surface of the base material (3) a thermosensitive coloring solution in which a color former and a developer are dispersed in a resin binder. The base material (3) is colored dark or black due to a chemical reaction that occurs when one or both of the color former and the developer are melted and brought into contact with each other by the heating during an autoclave process. The thermosensitive coloring layer preferably has an irreversible thermosensitive coloring property.

The color former is not particularly limited as long as it is generally used for a thermosensitive color former composition. Examples thereof include a fluorane compound, a triarylmethane compound, a spiro compound, a diphenylmethane compound, a thiazine compound, a lactam compound, a fluorene compound and the like.

The developer is not particularly limited as long as it is generally used for a thermosensitive color former composition. Examples thereof include an inorganic developer such as acid clay, activated clay, attapulgite, zeolite, bentonite, silica and aluminum silicate, and an organic developer such as a phenolic compound, a thiophenolic compound, a thiourea derivative, an organic acid and a metal salt thereof.

The resin used for the resin binder is not particularly limited as long as it is generally used for a thermosensitive color former composition. Examples thereof include polyvinyl alcohol, starch or a derivative thereof a cellulose derivative such as methoxycellulose, hydroxyethylcellulose, carboxymethylcellulose, methylcellulose and ethylcellulose; a water soluble macromolecule such as sodium polyacrylate, polyvinylpyrrolidone, acrylamide-acrylic acid ester copolymer, acrylamide-acrylic acid ester-methacrylic acid terpolymer, an alkali salt of styrene-maleic anhydride copolymer, an alkali salt of isobutylene-maleic anhydride copolymer, polyacrylamide, sodium alginate, gelatin and casein; an emulsion such as polyvinyl acetate, polyurethane, polyacrylic acid, polyacrylic acid ester, polymethacrylic acid ester, polybutyl methacrylate, vinyl chloride-vinyl acetate copolymer, and ethylene-vinyl acetate copolymer; a latex such as styrene-butadiene copolymer and styrene-butadiene-acrylic copolymer. These resins may be used alone or in combination of two or more.

Further, in the thermosensitive coloring layer, a conventional additive component such as a sensitizer such as stearamide, a filler, a surfactant, a lubricant, a pressure coloring inhibitor or the like may be used together, as long as the effects of the present invention are not impaired.

The amount of the color former is preferably 1% by weight or more and 50% by weight or less, more preferably 5% by weight or more and 30% by weight or less. Further, the amount of the developer is preferably 1% by weight or more and 70% by weight or less, more preferably 10% by weight or more and 50% by weight or less. The percent by weight of each component is a weight ratio of each component to the thermosensitive coloring layer.

The amount of the developer is preferably 1 part by weight or more and 20 parts by weight or less, more preferably 2 parts by weight or more and 10 parts by weight or less, with respect to 1 part by weight of the color former. These color formers and color developers may be used alone or in combination of two or more.

The base material (3) is not particularly limited. Applicable as the base material (3) are, for example, a paper base material such as high quality paper, kraft paper, glassine paper, art paper, coated paper, and moisture proof processed paper; an olefinic film base material such as polyethylene and polypropylene; a polyester film base material such as polyethylene terephthalate; a transparent or colored plastic film base material such as cellophane, polyvinylidene chloride, polyvinylidene chloride copolymer, polyvinylidene fluoride, polyvinyl chloride, and nylon; porous types of these plastic film base materials (including foamed base materials); a metal foil base material such as copper, aluminum and stainless steel; a composite sheet base material in which a metal foil and a plastic film are laminated; a vapor deposition foil base material in which aluminum or silica is vapor deposited; a non-woven fabric base material; and the like.

The thickness of the base material layer (4) as described above is not particularly limited, but is preferably 15 μm or more and 100 μm or less, more preferably 30 μm or more and 80 μm or less. In the case that the thickness is less than 15 μm, the flexibility becomes too high and the handleability as a label is deteriorated. On the other hand, in the case that the thickness exceeds 100 μm, the flexibility is deficient and the suitability for application is lowered.

### (Adhesive Layer)

The adhesive composition contained in the adhesive layer (5) is not particularly limited, and may be any one of an acrylic adhesive composition, a polyester adhesive composition, a polyurethane adhesive composition, a rubber adhesive composition, a silicone adhesive composition, and the like. Further, the adhesive composition may be any one of an emulsion type adhesive composition, a solvent type adhesive composition, and a solventless type adhesive composition, and may be any one of a crosslinked type adhesive composition and a non-crosslinked type adhesive composition. Conventionally known adhesive compositions may be used suitably.

### Third Embodiment

Mainly different points from the contents described in the first and second embodiments are specifically demonstrated hereinafter. In the information protection label (1) of the present invention, the base material (3) preferably exhibits solubility by an autoclave process. The base material (3) is dissolved due to a heated steam generated during an autoclave process, so that the information becomes visually unrecognizable.

The base material (3) which exhibits solubility by an autoclave process, includes, for example, water soluble paper, thermal water soluble paper and the like. The water soluble paper is not particularly limited, but includes, for example, water soluble paper which is made by mixing carboxymethyl cellulose fibers as the main fiber component, with water soluble natural or synthetic fibers, a water soluble glue or the like; and a water soluble polyvinyl alcohol resin sheet, an alginic acid sheet, and the like. Commercially available water soluble papers may be used.

### Fourth Embodiment

Mainly different points from the contents described in the first to third embodiments are specifically demonstrated hereinafter. In the information protection label (1) of the present invention, the base material (3) is preferably foamed by an autoclave process. The base material (3) expands due to the heating during the autoclave process, so that the information becomes visually unrecognizable. The base material (3) to be foamed by the autoclave process is not particularly limited. Examples thereof include a resin such as an ethylene-vinyl acetate copolymer, a polyethylene, a polypropylene, a polyester, a polyvinyl butyral, a polyvinyl chloride, a polyamide and a polyketone, and specifically include a resin material such as a rubber such as styrene-butadiene rubber (SBR) and polybutadiene rubber (BR). One, two or more types of the above base materials may be appropriately selected and used. An ethylene-vinyl acetate copolymer is preferably used. By using an ethylene-vinyl acetate copolymer, the expansion ratio can be increased. Moreover, the base material (3) preferably contains a foaming agent which exhibits foamability. The same foaming agents as described above may be used as the foaming agent.

### Fifth Embodiment

In the information protection label (1) of the present invention, the information display part (2) is preferably printed with an ink exhibiting a water disintegratability. A coloring material of the ink exhibiting a water disintegratability exudes due to a steam generated during an autoclave process, so that the information becomes visually unrecognizable. Mainly different points from the contents described in the first to fourth embodiments are specifically demonstrated hereinafter.

The ink exhibiting a water disintegratablility is not particularly limited, but, for example, an aqueous inkjet ink is preferable. Either an aqueous pigment ink or an aqueous dye ink may be used as an aqueous inkjet ink. When using an aqueous inkjet ink, it is preferable to use the base material layer (4) provided with the ink receiving layer (6) from a viewpoint of an ink fixing property.

### Sixth Embodiment

As shown in FIG. 2, it is preferable that the information protection label (1) of this embodiment comprises a base material layer (4) comprising an information display part (2) for displaying information and a base material (3), and an adhesive layer (5), wherein the base material layer (4) further comprises an ink receiving layer (6) exhibiting a water disintegratability by a autoclave process, between the base material (3) and the information display part (2). The ink receiving layer (6) is disintegrated due to a steam generated during an autoclave process, so that the information becomes visually unrecognizable. Mainly different points from the contents described in the first to fifth embodiments are specifically demonstrated hereinafter.

Examples of the ink receiving layer (6) include one consisting of a filler and a water soluble macromolecule, one consisting of a filler and a water soluble pulp, and the like. Examples of the filler include silica (silicon dioxide), kaolin, calcined clay, talc, calcium carbonate, barium sulfate, titanium dioxide, calcium carbonate and the like. Examples of the water soluble macromolecule include a polyvinyl alcohol; a saponified product of a copolymer of vinyl acetate and an unsaturated carboxylic acid such as acrylic acid, maleic acid, fumaric acid, itaconic acid, and allyl acetate; a polyvinyl alcohol resin such as an esterified product derived from an acid anhydride such as succinic anhydride, phthalic anhydride and maleic anhydride; a polyacrylamide; a sodium polyacrylate; a polyvinyl pyrrolidone; a cellulosic resin such as an acetylcellulose, an acetylbutylcellulose, a carboxymethylcellulose, a methylcellulose, a hydroxyethylcellulose, and a hydroxypropylcellulose; starch, dextrin, gelatin, animal glue, sodium alginate and the like. These fillers and water soluble macromolecules may be used alone or in combination of two or more.

The ink receiving layer (6) is applied and dried on the base material (3) by a blade coater, a bar coater, a roll coater, a size press or the like, with a thickness of 3 μm or more and 30 μm or less.

### Seventh Embodiment

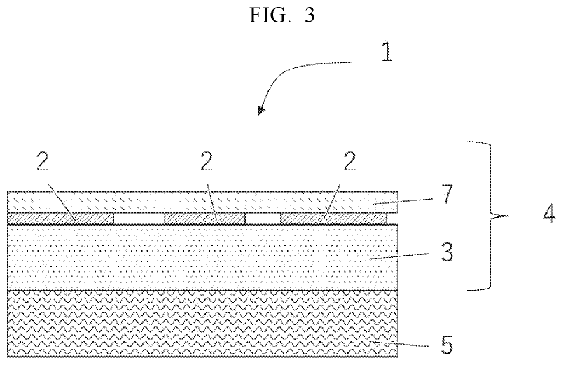

As shown in FIG. 3, it is preferable that the information protection label (1) of this embodiment comprises a base material layer (4) comprising an information display part (2) for displaying information and a base material (3), and an adhesive layer (5), wherein a transparent or translucent laminate base material (7) is provided on the surface of the information display part (2), and the laminate base material (7) has a property of developing a dark color or black color by an autoclave process. In FIG. 3, the laminate base material (7) is laminated on the surface of the information display part (2) via an adhesive layer (not shown) or the like. The laminate base material (7) develops a dark or black color due to the heating during an autoclave process, so that the information becomes visually unrecognizable. Mainly different points from the contents described in the first to sixth embodiments are specifically demonstrated hereinafter.

Examples of the laminate base material (7) having a property of developing a dark color or black color by an autoclave process, include a thermal film in which at least one thermosensitive coloring layer is formed on the laminate base material (7), and the like. The same thermosensitive coloring layer as described above may be used for the thermosensitive coloring layer.

The transparent or translucent laminate base material (7) is not particularly limited as long as it is a transparent or translucent resin film. Examples thereof include a polypropylene film, a polyethylene film, a polyvinylidene chloride film, a polyvinyl alcohol film, a polyester film, a polycarbonate film, a polyacrylonitrile film, a polystyrene film, an ethylene vinyl acetate copolymer film, an ethylene vinyl alcohol copolymer film, a polycarbonate film, a nylon film and the like. It is preferable that the haze value is 10% or less from a viewpoint of visibility of the information. These laminate base materials may be used alone or in combination of two or more.

The total light transmittance of the laminate base material (7) that is colored dark or black by the autoclave process at 121° C. for 20 min, is preferably 10% or less, more preferably 7% or less, from a viewpoint of invisibility. The total light transmittance can be determined, for example, by measurement according to the standard: ISO 13468-1 (similar to JIS K 7361-1) after autoclaving the laminate base material on which the thermosensitive coloring layer is formed.

Conventional methods may be uses as a method of laminating a laminate base material (7) on the surface of the information display part (2), to form the information protection label (1) of this embodiment.

### (Medical Label)

The information protection label (1) of the present invention is preferably used as a medical label, because it uses a material which changes in quality so that the information becomes visually unrecognizable by an autoclave process. It is because a medical label is often subjected to an autoclave process for a sterilization disposal operation, and the information becomes visually unrecognizable simultaneously with the sterilization.

### (Product)

The information protection label (1) of the present invention may be used by pasting it on a product. The product is not particularly limited, but examples of the product include, for example, a paper product such as a postal item, an invoice, a specification, a delivery notice and a transmittal letter; an article such as a food, a medicine, a biological object, an industrial product, an artistic handicraft and a miscellaneous good; a packaging container that stores the article, and the like. Here, the packaging container may have any shape such as a bag shape and a box shape.

The product of the present invention is preferably any one of a biological sample container, a medical container and a medical device. It is because these products are often subjected to an autoclave process for a sterilization disposal operation, and necessity of the information protection is high.

The biological sample container is a container for stowing or storing a biological sample used for academic research, examination, treatment or the like. The biological sample is not particularly limited, but examples thereof include, for example, an animal cell line including a human cell line, a plant cell line, a fungi, a bacteria, a virus, primary cultured cells, a body fluid of an animal including a human such as blood, serum, urine, saliva and mucus, a lymph fluid, a spinal fluid, a bone marrow fluid, a tissue fragment, a tissue extract and the like.

The medical container is not particularly limited, but examples thereof include, for example, an infusion container such as an infusion bags and an infusion bottle, a medicinal solution container, an IVH bag storing nutrients for direct administration to the central vein, a bag containing enteral nutrients, a urine collection bag, a blood bag, and a packaging container for a medical device.

The medical device is not particularly limited, but examples thereof include, for example, a syringe, a catheter, an endoscope, a stent, an artificial cardiopulmonary extracorporeal circuit, a plate, a petri dish, a dish, a flask, a tube, an intravascular drug delivery port system, a surgical suture material, a drug delivery system, an implantable prosthetic lead and electrode, an artificial heart valve, a venous valve, another implant, a blood oxygenator, a blood pump, a blood sensor, a detector, a blood tube, a renal dialysis device, a dialysis membrane, a detoxification cartridge, an artificial cardiopulmonary device, a dental device, and the like.

The present inventions are demonstrated using embodiments of the present inventions as mentioned above. The technical scope of the present inventions is not limited to those embodiments. Further, it is apparent to a person skilled in the art that a various change or modification can be added to those embodiments, and those various changed or modified embodiments are included to the technical scope of the present inventions.

### REFERENCE SIGNS LIST

1 information protection label
2 information display part
3 base material
4 base material layer
5 adhesive layer
6 ink receiving layer
7 transparent or translucent laminate base material

1. An information protection label, comprising:
a base material layer comprising an information display part for displaying information and a base material which is chemically or physically deformable, and
an adhesive layer,
wherein the base material layer comprises a material which changes in quality so that the information becomes visually unrecognizable by an autoclave process.

2. The information protection label according to claim 1,
wherein the base material is a shrinkable film;
the adhesive layer is composed of an adhesive composition whose adhesive strength is reduced by an autoclave process;
an adhesive strength (A1) of the information protection label to a polypropylene plate 24 hours after pasting and before an autoclave process, is 2N/25 mm or more;
an adhesive strength (A2) of the information protection label to a polypropylene plate after the information protection label is autoclaved at 121° C. for 20 min is 1 N/25 mm or less; and
the ratio ((A2)/(A1)) of the adhesive strengths (A1) and (A2) is 0.0001 or more and 0.5 or less.

3. The information protection label according to claim 2, wherein the adhesive composition whose adhesive strength is reduced by an autoclave process, is a rubber based adhesive, a thermosetting adhesive, a water soluble adhesive, or a heat foamable adhesive.

4. The information protection label according to claim 1, wherein the base material develops a dark color or black color by an autoclave process.

5. The information protection label according to claim 4, wherein the base material is a thermal coloring paper.

6. The information protection label according to claim 1, wherein the base material is water soluble paper, and exhibits solubility by an autoclave process.

7. The information protection label according to claim 1, wherein the base material comprises a foaming agent, and is foamed by an autoclave process.

8. The information protection label according to claim 1, wherein the information display part is printed with an ink exhibiting a water disintegratability.

9. The information protection label according to claim 1, wherein the base material layer comprises an ink receiving layer exhibiting a water disintegratability by an autoclave process.

10. The information protection label according to claim 1,
wherein a transparent or translucent laminate base material is provided on the surface of the information display part, and
the laminate base material has a property of developing a dark color or black color by an autoclave process.

11. A product which the information protection label according to claim 1 is pasted on.

12. A product which the information protection label according to claim 2 is pasted on.

13. A product which the information protection label according to claim 3 is pasted on.

14. A product which the information protection label according to claim 4 is pasted on.

15. A product which the information protection label according to claim 5 is pasted on.

16. A product which the information protection label according to claim 6 is pasted on.

17. A product which the information protection label according to claim 8 is pasted on.

18. A product which the information protection label according to claim 9 is pasted on.

19. A product which the information protection label according to claim 10 is pasted on.

20. The product according to claim 11, wherein the product is any one of a biological sample container, a medical container and a medical device.
